(19) United States
(12) Patent Application Publication
(10) Pub. No.: US 2026/0177544 A1
(43) Pub. Date: Jun. 25, 2026

(54) METHOD FOR DETECTING TEST SUBSTANCE IN SPECIMEN

(71) Applicant: TOKYO METROPOLITAN INSTITUTE FOR GERIATRICS AND GERONTOLOGY, Tokyo (JP)

(72) Inventors: Tetsuya KIMURA, Tokyo (JP); Katsumi KAWANO, Tokyo (JP)

(73) Assignee: TOKYO METROPOLITAN INSTITUTE FOR GERIATRICS AND GERONTOLOGY, Tokyo (JP)

(21) Appl. No.: 19/123,474

(22) PCT Filed: Oct. 24, 2023
(86) PCT No.: PCT/JP2023/038256

§ 371 Date: Apr. 23, 2025

(30) Foreign Application Priority Data
Oct. 24, 2022 (JP) 2022-170188

(51) Int. Cl. G01N 33/543; G01N 21/39; G01N 33/532

(52) U.S. Cl. CPC G01N 33/54326; G01N 21/39; G01N 33/532; G01N 2446/00; G01N 2470/06; G01N 2470/10

(57) ABSTRACT

An object of the present invention is to provide a highly sensitive method for detecting a biomarker. The object can be achieved by a method for detecting a test substance in a specimen of the present invention, the method being (A) a sandwich method including: (1) bringing a specimen into contact with a magnetic particle on which a primary substance is immobilized and which has a particle diameter that does not cause interference with a wavelength for measurement with a laser, to form a primary substance-test substance complex of the primary substance and a test substance in the specimen; (2) bringing the primary substance-test substance complex into contact with a secondary substance, to form a primary substance-test substance-secondary substance complex of the primary substance-test substance complex and the secondary substance; and (3) detecting the primary substance-test substance-secondary substance complex on the magnetic particle with a laser; or (B) a competitive method including: (1) bringing a specimen and a secondary substance into contact with a magnetic particle on which a primary substance is immobilized and which has a particle diameter that does not cause interference with a wavelength for measurement with a laser, to form a primary substance-secondary substance complex of the primary substance and the secondary substance; and (2) detecting the primary substance-secondary substance complex on the magnetic particle with a laser.

### TECHNICAL FIELD

The present invention relates to a method for detecting a test substance in a specimen. According to the present invention, single molecular counting (which may hereinafter be referred to as SMC) can be performed in a solid phase.

### BACKGROUND ART

Single-molecular array (SIMOA) technology and single-molecular count (SMC) technology are known as the most sensitive methods for biomarker detection. However, the concentration of pathologically important molecules, such as phosphorylated tau, in blood is extremely low, and a prior concentration process is reportedly required to detect such molecules (Non-Patent Literature 1).

### CITATION LIST

### Patent Literature

Non-Patent Literature 1: “Alzheimer's & Dementia” (United States) 2021, vol. 13, e12204

### SUMMARY OF INVENTION

### Technical Problem

Thus, an object of the present invention is to provide a highly sensitive method for detecting a biomarker.

### Solution to Problem

As a result of diligent studies on a highly sensitive method for detecting a biomarker, the present inventors have surprisingly found that when an ultrafine bead with less influence of Mie dissipation is used as a carrier in SMC technology, a phosphor attached to the bead can be identified without losing the magnetic adsorption property of the bead. It has also been found that molecular counting can be performed even in a solid phase, which is a bead aggregate. It has also been found that a high sensitivity can be achieved without performing a washing step.

The present invention is based on these findings.

Thus, the present invention relates to:
[1] a method for detecting a test substance in a specimen, the method being
(A) a sandwich method including:
(1) bringing a specimen into contact with a magnetic particle on which a primary substance is immobilized and which has a particle diameter that does not cause interference with a wavelength for measurement with a laser, to form a primary substance-test substance complex of the primary substance and a test substance in the specimen;
(2) bringing the primary substance-test substance complex into contact with a secondary substance, to form a primary substance-test substance-secondary substance complex; and
(3) detecting the primary substance-test substance-secondary substance complex on the magnetic particle with a laser;
(B) a sandwich method including:
(1) bringing a primary substance into contact with a test substance in a specimen and a secondary substance, to form a primary substance-test substance-secondary substance complex;
(2) binding the primary substance-test substance-secondary substance complex to a magnetic particle having a particle diameter that does not cause interference with a wavelength for measurement with a laser; and
(3) detecting the primary substance-test substance-secondary substance complex on the magnetic particle with a laser;
(C) a competitive method including:
(1) bringing a specimen and a secondary substance into contact with a magnetic particle on which a primary substance is immobilized and which has a particle diameter that does not cause interference with a wavelength for measurement with a laser, to form a primary substance-secondary substance complex of the primary substance and the secondary substance; and
(2) detecting the primary substance-secondary substance complex on the magnetic particle with a laser; or
(D) a competitive method including:
(1) bringing a primary substance into contact with a specimen and a secondary substance, to form a primary substance-secondary substance complex of the primary substance and the secondary substance;
(2) binding the primary substance-secondary substance complex to a magnetic particle having a particle diameter that does not cause interference with a wavelength for measurement with a laser; and
(3) detecting the primary substance-secondary substance complex on the magnetic particle with a laser;
[2] the method for detecting a test substance according to [1], wherein, in the sandwich method (A) or (B), the primary substance is a capture antibody, the test substance is an antigen in the specimen, and the secondary substance is a detection antibody;
[3] the method for detecting a test substance according to [1], wherein, in the sandwich method (A) or (B), the primary substance is an antigen, the test substance is an antibody in the specimen, and the secondary substance is a detection antibody;
[4] the method for detecting a test substance according to [1], wherein, in the competitive method (C) or (D), the primary substance is a capture antibody, the test substance is an antigen or antibody in the specimen, and the secondary substance is a standard antigen;
[5] the method for detecting a test substance according to [1], wherein, in the competitive method (C) or (D), the primary substance is an antigen, the test substance is an antigen or antibody in the specimen, and the secondary substance is a detection antibody;
[6] the method for detecting a test substance according to any of [1] to [5], wherein the detection with a laser uses a laser confocal microscope;
[7] the method for detecting a test substance according to any of [1] to [5], wherein the particle diameter is from 20 to 500 nm;
[8] a magnetic particle having a particle diameter that does not cause interference with a wavelength for measurement with a laser, the magnetic particle being for use in a method for detecting a test substance in a specimen, the method being a sandwich method (A) or (B), or a competitive method (C) or (D), the method including steps described below:
(A) a sandwich method including:
(1) bringing a specimen into contact with the magnetic particle on which a primary substance is immobilized, to form a primary substance-test substance complex of the primary substance and a test substance in the specimen;
(2) bringing the primary substance-test substance complex into contact with a secondary substance, to form a primary substance-test substance-secondary substance complex; and
(3) detecting the primary substance-test substance-secondary substance complex on the magnetic particle with a laser;
(B) a sandwich method including:
(1) bringing a primary substance into contact with a test substance in a specimen and a secondary substance, to form a primary substance-test substance-secondary substance complex;
(2) binding the primary substance-test substance-secondary substance complex to the magnetic particle; and
(3) detecting the primary substance-test substance-secondary substance complex on the magnetic particle with a laser;
(C) a competitive method including:
(1) bringing a specimen and a secondary substance into contact with the magnetic particle on which a primary substance is immobilized, to form a primary substance-secondary substance complex of the primary substance and the secondary substance; and
(2) detecting the primary substance-secondary substance complex on the magnetic particle with a laser; or
(D) a competitive method including:
(1) bringing a primary substance into contact with a specimen and a secondary substance, to form a primary substance-secondary substance complex of the primary substance and the secondary substance;
(2) binding the primary substance-secondary substance complex to the magnetic particle; and
(3) detecting the primary substance-secondary substance complex on the magnetic particle with a laser;
[9] the magnetic particle according to [8], wherein, in the sandwich method (A) or (B), the primary substance is a capture antibody, the test substance is an antigen in the specimen, and the secondary substance is a detection antibody;
[10] the magnetic particle according to [8], wherein, in the sandwich method (A) or (B), the primary substance is an antigen, the test substance is an antibody in the specimen, and the secondary substance is a detection antibody;
[11] the magnetic particle according to [8], wherein, in the competitive method (C) or (D), the primary substance is a capture antibody, the test substance is an antigen or antibody in the specimen, and the secondary substance is a standard antigen;
[12] the magnetic particle according to [8], wherein, in the competitive method (C) or (D), the primary substance is an antigen, the test substance is an antigen or antibody in the specimen, and the secondary substance is a detection antibody;
[13] the magnetic particle according to any of [8] to [12], wherein the detection with a laser uses a laser confocal microscope; and
[14] the magnetic particle according to any of [8] to [12], wherein the particle diameter is from 20 to 500 nm.

### Advantageous Effects of Invention

According to the detection method and the magnetic particle of the present invention, a biomarker can be detected with high sensitivity.

### BRIEF DESCRIPTION OF DRAWINGS

FIG. 1 schematically illustrates a liquid phase method (A), which is single molecule detection (SMC) known in the art, and a solid phase method (B), which is single molecule detection (SMC) of the present invention.

FIG. 2 is a graph illustrating measurement of tau protein by an SMC method in Example 1 and measurement of tau protein by an SMC method in Comparative Example 1.

FIG. 3 is a graph illustrating measurement of tau protein in plasma by the SMC method of the present invention.

FIG. 4 is graphs illustrating interference of magnetic particles having a particle diameter of 100 nm and magnetic particles having a particle diameter of 1 μm with a measurement wavelength.

FIG. 5 is a graph illustrating measurement of tau protein in plasma by the SMC method, i.e., a sandwich method (B) of the present invention.

### DESCRIPTION OF EMBODIMENTS

The method for detecting a test substance in a specimen of the present invention is a sandwich method or a competitive method. The sandwich method is a method (A) including:
(1) bringing a specimen into contact with a magnetic particle on which a primary substance is immobilized and which has a particle diameter that does not cause interference with a wavelength for measurement with a laser, to form a primary substance-test substance complex of the primary substance and a test substance in the specimen;
(2) bringing the primary substance-test substance complex into contact with a secondary substance, to form a primary substance-test substance-secondary substance complex; and
(3) detecting the primary substance-test substance-secondary substance complex on the magnetic particle with a laser; or a method (B) including:
(1) bringing a primary substance into contact with a test substance in a specimen and a secondary substance, to form a primary substance-test substance-secondary substance complex;
(2) binding the primary substance-test substance-secondary substance complex to a magnetic particle having a particle diameter that does not cause interference with a wavelength for measurement with a laser; and
(3) detecting the primary substance-test substance-secondary substance complex on the magnetic particle with a laser. The competitive method is a method (C) including:
(1) bringing a specimen and a secondary substance into contact with a magnetic particle on which a primary substance is immobilized and which has a particle diameter that does not cause interference with a wavelength for measurement with a laser, to form a primary substance-secondary substance complex of the primary substance and the secondary substance; and
(2) detecting the primary substance-secondary substance complex on the magnetic particle with a laser; or a method (D) including:
(1) bringing a primary substance into contact with a specimen and a secondary substance, to form a primary substance-secondary substance complex of the primary substance and the secondary substance;
(2) binding the primary substance-secondary substance complex to a magnetic particle having a particle diameter that does not cause interference with a wavelength for measurement with a laser; and
(3) detecting the primary substance-secondary substance complex on the magnetic particle with a laser.

### Magnetic Particle

The magnetic particle used in the present invention has a particle diameter that does not cause interference with a wavelength for measurement with a laser. The particle diameter that does not cause interference with a wavelength for measurement with a laser is not particularly limited as long as the effects of the present invention can be obtained. The particle diameter is, for example, from 20 to 500 nm. The lower limit of the particle diameter is 30 nm or more in an embodiment, 40 nm or more in another embodiment, and 50 nm or more in yet another embodiment. The upper limit of the particle diameter is 400 nm or less in an embodiment, 300 nm or less in another embodiment, and 200 nm or less in yet another embodiment. The lower limit and the upper limit can be appropriately combined to provide a range of the particle diameter of the magnetic particle. Although not limited, the magnetic particle having a particle diameter in the above range does not cause interference with a wavelength for measurement with a laser.

### (A) Sandwich Method

### (1) Primary Substance-Test Substance Complex Formation Step

In the primary substance-test substance complex formation step of the sandwich method (A), a specimen is brought into contact with a magnetic particle on which a primary substance is immobilized and which has a particle diameter that does not cause interference with a wavelength for measurement with a laser, to form a primary substance-test substance complex of the primary substance and a test substance in the specimen.

Examples of the test substance in the specimen include, but are not particularly limited to, an antigen (e.g., a protein, a sugar, or a lipid) or an antibody (e.g., an antigen-specific human antibody).

The primary substance is not particularly limited as long as it can bind to a test substance (e.g., an antigen or an antibody) in the specimen. Examples of the primary substance include an antibody, an antigen, a lectin, a receptor, or a sugar chain. When an antibody is used, the sandwich method is an immunoassay.

For the binding between the primary substance and the magnetic particle, any method commonly used in this field can be used without limitation. Examples of the binding include hydrophobic interaction (physical adsorption), avidin-biotin binding, or covalent binding. In the case of avidin-biotin binding, for example, avidin may be bound to the magnetic particle, and biotin may be bound to the primary substance. On the contrary, biotin may be bound to the magnetic particle, and avidin may be bound to the primary substance.

When the primary substance is an antibody and the test substance is an antigen in the specimen, the primary substance-test substance complex is an immune complex of the antibody and the antigen. Also, when the primary substance is an antigen and the test substance is an antibody in the specimen, the primary substance-test substance complex is an immune complex of the antibody and the antigen.

### (2) Primary Substance-Test Substance-Secondary Substance Complex Formation Step

In the primary substance-test substance-secondary substance complex formation step of the sandwich method (A), the primary substance-test substance complex is brought into contact with a secondary substance, to form a primary substance-test substance-secondary substance complex of the primary substance-test substance complex and the secondary substance.

The secondary substance is not particularly limited as long as it can bind to a test substance (e.g., an antigen or an antibody) in the primary substance-test substance complex. Examples of the secondary substance include an antibody, an antigen, a lectin, a receptor, or a sugar chain. When an antibody is used, the sandwich method is an immunoassay.

The secondary substance is not limited, but is preferably labeled. Examples of the labeling substance include, but are not particularly limited to, a luminescent substance (e.g., an acridinium derivative) or a fluorescent substance (fluorescein, rhodamine, dansyl chloride, fluoronitrobenzofurazan, a europium chelate, or a samarium chelate). A fluorescent substance is preferred. When the secondary substance is directly labeled, the primary substance-test substance-secondary substance complex can be detected in the primary substance-test substance-secondary substance complex detection step.

However, even in the case where the secondary substance is not directly labeled, when an antibody against the secondary substance is labeled, and the labeled antibody is bound to the secondary substance to form a labeled complex, the labeled complex can be detected in the primary substance-test substance-secondary substance complex detection step. Furthermore, when the secondary substance is labeled with biotin, and the labeled secondary substance is bound to avidin labeled with the fluorescent substance to form a labeled complex, the labeled complex can be detected in the primary substance-test substance-secondary substance complex detection step.

### (3) Primary Substance-Test Substance-Secondary Substance Complex Detection Step

In the primary substance-test substance-secondary substance complex detection step of the sandwich method (A), the primary substance-test substance-secondary substance complex on the magnetic particle is detected with a laser.

The detection with a laser is not particularly limited, but is preferably detection with a laser confocal microscope.

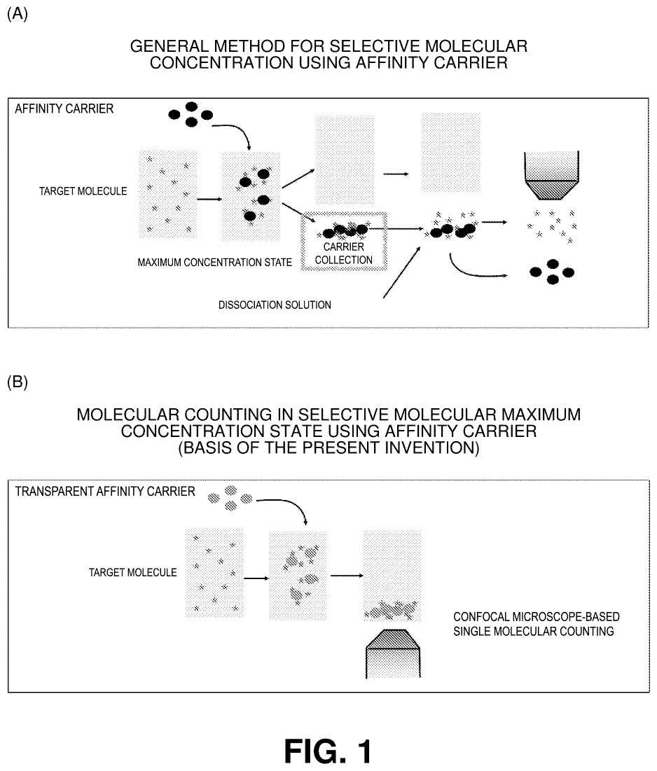

Usually, in detection by SMC, as illustrated in FIG. 1(A), the labeling substance of the primary substance-test substance-secondary substance complex is dissociated from magnetic particles and detected in a liquid phase. However, in the present invention, as illustrated in FIG. 1(B), magnetic particles can be collected with a magnet and detected in the form of pellets in a solid phase.

The measurement wavelength in the single molecule measurement is not particularly limited, but is, for example, from 100 to 1000 nm, preferably from 200 to 900 nm, more preferably from 300 to 800 nm, and even more preferably from 400 to 700 nm.

In the sandwich method (A), examples of the test substance include, but are not limited to, an antigen or an antibody. An example of an embodiment of an antigen detection method and an antibody detection method will be described below.

### Antigen Detection Method

In the antigen detection method, for example, the primary substance is a capture antibody, the test substance is an antigen in a specimen, and the secondary substance is a detection antibody.

The antibody (capture antibody) that binds to the antigen is immobilized on the magnetic particle. For example, in the case of immobilization using avidin-biotin, the capture antibody can be immobilized on the magnetic particle through avidin-biotin binding by, for example, binding avidin to the magnetic particle, binding biotin to the capture antibody, and mixing these. Subsequently, to prevent non-specific adsorption to the capture antibody and the magnetic particle, the magnetic particle is blocked with an appropriate blocking agent (e.g., bovine serum albumin or gelatin). A test sample containing the antigen is added together with a primary reaction liquid to the magnetic particle on which the capture antibody is immobilized, and the capture antibody is brought into contact with and bound to the antigen (primary substance-test substance complex formation step). Thereafter, the antigen not bound to the capture antibody and a contaminant are washed with an appropriate washing liquid (e.g., a phosphate buffer containing a surfactant). Next, a detection antibody (labeled antibody) in which an antibody that binds to a captured antigen is bound to a labeling substance is added, and the detection antibody is bound to the captured antigen (primary substance-test substance-secondary substance complex formation step). This reaction forms an immune complex of the capture antibody-antigen-detection antibody on the magnetic particle. Unbound detection antibody is washed with a washing liquid. The magnetic particle is collected with a magnet and detected with a laser (primary substance-test substance-secondary substance complex detection step). It is also possible to label an antibody that binds to the detection antibody and detect a signal without directly labeling the detection antibody.

### Antibody Detection Method

In the antibody detection method, for example, the primary substance is an antigen, the test substance is an antibody in the specimen, and the secondary substance is a detection antibody.

The antigen is immobilized on the magnetic particle. For example, in the case of immobilization using avidin-biotin, the antigen can be immobilized on the magnetic particle through avidin-biotin binding by, for example, binding avidin to the magnetic particle, binding biotin to the antigen, and mixing these. Subsequently, to prevent non-specific adsorption to the antigen and the magnetic particle, the magnetic particle is blocked with an appropriate blocking agent (e.g., bovine serum albumin or gelatin). A test sample containing the antibody against the antigen is added together with a primary reaction liquid to the magnetic particle on which the antigen is immobilized, and the antigen is brought into contact with and bound to the antibody in the test sample (primary substance-test substance complex formation step). Thereafter, the antibody not bound to the antigen and a contaminant are washed with an appropriate washing liquid (e.g., a phosphate buffer containing a surfactant). Next, a detection antibody (labeled antibody) in which an antibody that binds to a captured antibody is bound to a labeling substance is added, and the detection antibody is bound to the captured antibody (primary substance-test substance-secondary substance complex formation step). This reaction forms an immune complex of the antigen-antibody in the specimen-detection antibody on the magnetic particle. Unbound detection antibody is washed with a washing liquid. The magnetic particle is collected with a magnet and detected with a laser (primary substance-test substance-secondary substance complex detection step). It is also possible to label an antibody that binds to the detection antibody and detect a signal without directly labeling the detection antibody.

The sandwich method (A) may be carried out without carrying out the washing operation in the sandwich method (A).

### (B) Sandwich Method

### (1) Primary Substance-Test Substance-Secondary Substance Complex Formation Step

In the primary substance-test substance-secondary substance complex formation step of the sandwich method (B), a primary substance is brought into contact with a test substance in a specimen and a secondary substance, to form a primary substance-test substance-secondary substance complex. For the test substance in the specimen and the primary substance, the test substance and the primary substance described in “(1) Primary Substance-Test Substance Complex Formation Step” of the sandwich method (A) can be used. For the secondary substance, the secondary substance described in “(2) Primary Substance-Test Substance-Secondary Substance Complex Formation Step” of the sandwich method (A) can be used.

### (2) Primary Substance-Test Substance-Secondary Substance Complex Binding Step

In the primary substance-test substance-secondary substance complex binding step of the sandwich method (B), the primary substance-test substance-secondary substance complex is bound to a magnetic particle having a particle diameter that does not cause interference with a wavelength for measurement with a laser. The binding between the magnetic particle and the primary substance-test substance-secondary substance complex is not particularly limited as long as the specificity can be secured. That is, the magnetic particle can be specifically bound to the primary substance-test substance-secondary substance complex by binding two substances that specifically bind to each other to the magnetic particle and the complex, respectively, followed by mixing. Examples of the two substances that specifically bind to each other include avidin and biotin. That is, the magnetic particle can be specifically bound to the complex by, for example, binding avidin to the magnetic particle and binding biotin to the complex. Biotin bound to the complex is preferably bound to, for example, the primary substance or the secondary substance. For example, when biotin is bound to the primary substance, a labeling substance for detection is preferably bound to the secondary substance. On the contrary, a labeling substance may be bound to the primary substance, and biotin may be bound to the secondary substance.

For the two substances that specifically bind to each other, an antigen and an antibody, a lectin and a sugar chain, or a receptor and a ligand can be used instead of avidin and biotin.

### (3) Primary Substance-Test Substance-Secondary Substance Complex Detection Step

In the primary substance-test substance-secondary substance complex detection step of the sandwich method (B), the primary substance-test substance-secondary substance complex on the magnetic particle is detected with a laser. The primary substance-test substance-secondary substance complex detection step of the sandwich method (B) can be carried out in the same manner as the primary substance-test substance-secondary substance complex detection step described in the sandwich method (B).

Also in the sandwich method (B), examples of the test substance include, but are not limited to, an antigen or an antibody as in the sandwich method (A). The antigen detection method and the antibody detection method of the sandwich method (A) can be carried out in the same manner except that the order of binding between the magnetic particle and the primary substance or the like is different.

In the sandwich method (A), the measurement can be carried out without the washing operation. Meanwhile, in the sandwich method (B), after formation of the primary substance-test substance-secondary substance complex, the magnetic particle is bound to the complex. Thus, the measurement can be carried out without the washing operation more efficiently than in the case of the sandwich method (A).

### (C) Competitive Method

### (1) Primary Substance-Secondary Substance Complex Formation Step

In the primary substance-secondary substance complex formation step (1) of the competitive method (C), a specimen and a secondary substance are brought into contact with a magnetic particle on which a primary substance is immobilized and which has a particle diameter that does not cause interference with a wavelength for measurement with a laser, to form a primary substance-secondary substance complex of the primary substance and the secondary substance.

Examples of the test substance in the specimen include, but are not particularly limited to, an antigen (e.g., a protein, a sugar, or a lipid) or an antibody (e.g., an antigen-specific human antibody).

The primary substance is not particularly limited as long as it can bind to a secondary substance (e.g., an antigen or an antibody). Examples of the primary substance include an antibody, an antigen, a lectin, a receptor, and a sugar chain. When an antibody is used, the competitive method is an immunoassay.

For the binding between the primary substance and the magnetic particle, any method commonly used in this field can be used without limitation. Examples of the binding include hydrophobic interaction (physical adsorption), avidin-biotin binding, or covalent binding. In the case of avidin-biotin binding, for example, avidin may be bound to the magnetic particle, and biotin may be bound to the primary substance. On the contrary, biotin may be bound to the magnetic particle, and avidin may be bound to the primary substance.

When the primary substance is an antibody and the test substance is an antigen or antibody in the specimen, the primary substance-secondary substance complex is an immune complex of the antibody of the primary substance and the antigen of the secondary substance. When the primary substance is an antigen and the test substance is an antibody or antigen in the specimen, the primary substance-secondary substance complex is an immune complex of the antigen of the primary substance and the antigen of the secondary substance.

The secondary substance is not limited, but is preferably labeled. Examples of the labeling substance include, but are not particularly limited to, enzymes (e.g., horseradish peroxidase (HRP), alkaline phosphatase, β-galactosidase, and luciferase), a luminescent substance (e.g., an acridinium derivative), or a fluorescent substance (fluorescein, rhodamine, dansyl chloride, fluoronitrobenzofurazan, a europium chelate, or a samarium chelate). A fluorescent substance is preferred. When the secondary substance is directly labeled, the primary substance-secondary substance complex can be detected in the primary substance-secondary substance complex detection step.

However, even in the case where the secondary substance is not directly labeled, when an antibody against the secondary substance is labeled, and the labeled antibody is bound to the secondary substance to form a labeled complex, the labeled complex can be detected in the primary substance-secondary substance complex detection step. Furthermore, when the secondary substance is labeled with biotin, and the labeled secondary substance is bound to avidin labeled with the fluorescent substance to form a labeled complex, the labeled complex can be detected in the primary substance-secondary substance complex detection step.

### (2) Primary Substance-Secondary Substance Complex Detection Step

In the primary substance-secondary substance complex detection step of the competitive method, the primary substance-secondary substance complex on the magnetic particle is detected with a laser.

The detection with a laser is not particularly limited, but is preferably detection with a laser confocal microscope.

Usually, in detection by SMC, as illustrated in FIG. 1(A), the labeling substance of the primary substance-test substance complex is dissociated from magnetic particles and detected in a liquid phase. However, in the present invention, as illustrated in FIG. 1(B), magnetic particles can be collected with a magnet and detected in the form of pellets in a solid phase.

In the competitive method (C), examples of the test substance include, but are not limited to, an antigen or an antibody. Examples of the competitive method include, but are not limited to, an “antibody immobilization/antigen labeling method” in which an antibody (primary substance) is immobilized on a magnetic particle and an antigen (secondary substance) is labeled, and an “antigen immobilization/antibody labeling method” in which an antigen (primary substance) is immobilized on a magnetic particle and an antibody (secondary substance) is labeled. Embodiments of the methods will be described below.

### Antibody Immobilization/Antigen Labeling Method

In the antibody immobilization/antigen labeling method, the primary substance is a capture antibody, the test substance is an antigen or antibody in the specimen, and the secondary substance is a standard antigen.

The antibody (capture antibody) that binds to the antigen is immobilized on the magnetic particle. For example, in the case of immobilization using avidin-biotin, the capture antibody can be immobilized on the magnetic particle through avidin-biotin binding by, for example, binding avidin to the magnetic particle, binding biotin to the capture antibody, and mixing these. Subsequently, to prevent non-specific adsorption to the capture antibody and the magnetic particle, the magnetic particle is blocked with an appropriate blocking agent (e.g., bovine serum albumin or gelatin). A test sample containing an antigen or an antibody and a labeled standard antigen are added together with a primary reaction liquid to the magnetic particle on which the capture antibody is immobilized, and the capture antibody is brought into contact with and bound to the labeled standard antigen (primary substance-secondary substance complex formation step).

In the case of measuring the antigen in the test sample, the antigen is measured as follows. That is, in this step, in addition to the binding between the capture antibody and the labeled standard antigen, the binding between the capture antibody and the antigen in the test sample occurs, and these two bindings compete in the reaction system. When the amount of the antigen in the test sample is large, the binding between the capture antibody and the labeled standard antigen decreases, whereas when the amount of the antigen in the test sample is small, the binding between the capture antibody and the labeled standard antigen increases. The amount of the antigen in the test sample can be calculated by measuring a standard sample containing a known amount of the antigen and preparing a standard curve.

In the case of measuring the antibody in the test sample, the antibody is measured as follows. That is, in this step, in addition to the binding between the capture antibody and the labeled standard antigen, the binding between the antibody in the test sample and the labeled standard antigen occurs, and these two bindings compete in the reaction system. When the amount of the antibody in the test sample is large, the binding between the capture antibody and the labeled standard antigen decreases, whereas when the amount of the antibody in the test sample is small, the binding between the capture antibody and the labeled standard antigen increases. The amount of the antibody in the test sample can be calculated by measuring a standard sample containing a known amount of the antibody and preparing a standard curve.

Thereafter, the antigen not bound to the capture antibody and a contaminant are washed with an appropriate washing liquid (e.g., a phosphate buffer containing a surfactant). The reaction described above forms an immune complex of the capture antibody-labeled standard antigen on the magnetic particle. The magnetic particle is collected with a magnet and detected with a laser (primary substance-secondary substance complex detection step). It is also possible to label an antibody that binds to the detection antibody and detect a signal without directly labeling the detection antibody.

### Antigen Immobilization/Antibody Labeling Method

In the antigen immobilization/antibody labeling method, the primary substance is an antigen, the test substance is an antigen or antibody in the specimen, and the secondary substance is a detection antibody.

A standard antigen is immobilized on the magnetic particle. For example, in the case of immobilization using avidin-biotin, the standard antigen can be immobilized on the magnetic particle through avidin-biotin binding by, for example, binding avidin to the magnetic particle, binding biotin to the standard antigen, and mixing these. Subsequently, to prevent non-specific adsorption to the capture antibody and the magnetic particle, the magnetic particle is blocked with an appropriate blocking agent (e.g., bovine serum albumin or gelatin). A test sample containing an antigen or an antibody and a labeled detection antibody are added together with a primary reaction liquid to the magnetic particle on which the standard antigen is immobilized, and the standard antigen is brought into contact with and bound to the labeled detection antibody (primary substance-secondary substance complex formation step).

In the case of measuring the antigen in the test sample, the antigen is measured as follows. That is, in this step, in addition to the binding between the standard antigen and the labeled detection antibody, the binding between the antibody in the test sample and the labeled detection antibody occurs, and these two bindings compete in the reaction system. When the amount of the antigen in the test sample is large, the binding between the standard antigen and the labeled detection antibody decreases, whereas when the amount of the antigen in the test sample is small, the binding between the standard antigen and the labeled detection antibody increases. The amount of the antigen in the test sample can be calculated by measuring a standard sample containing a known amount of the antigen and preparing a standard curve.

In the case of measuring the antibody in the test sample, the antibody is measured as follows. That is, in this step, in addition to the binding between the standard antigen and the labeled detection antibody, the binding between the standard antigen and the antibody in the test sample occurs, and these two bindings compete in the reaction system. When the amount of the antibody in the test sample is large, the binding between the standard antigen and the labeled detection antibody decreases, whereas when the amount of the antibody in the test sample is small, the binding between the standard antigen and the labeled detection antibody increases. The amount of the antibody in the test sample can be calculated by measuring a standard sample containing a known amount of the antibody amount and preparing a standard curve.

Thereafter, the antigen not bound to the capture antibody and a contaminant are washed with an appropriate washing liquid (e.g., a phosphate buffer containing a surfactant). The reaction described above forms an immune complex of the standard antigen-labeled detection antibody on the magnetic particle. The magnetic particle is collected with a magnet and detected with a laser (primary substance-secondary substance complex detection step). It is also possible to label an antibody that binds to the detection antibody and detect a signal without directly labeling the detection antibody.

The competitive method (C) may be carried out without carrying out the washing operation in the competitive method (C).

### Competitive Method (D)

### (1) Primary Substance-Secondary Substance Complex Formation Step

In the primary substance-secondary substance complex formation step of the competitive method (D), a primary substance is brought into contact with a specimen and a secondary substance, to form a primary substance-secondary substance complex of the primary substance and the secondary substance.

For the primary substance, the test substance in the specimen, and the secondary substance, the primary substance, the test substance in the specimen, and the secondary substance described in “Primary Substance-Secondary Substance Complex Formation Step” of the competitive method (C) can be used.

### (2) Primary Substance-Secondary Substance Complex Binding Step

In the primary substance-secondary substance complex binding step of the competitive method (D), the primary substance-secondary substance complex is bound to a magnetic particle having a particle diameter that does not cause interference with a wavelength for measurement with a laser. The binding between the magnetic particle and the primary substance-secondary substance complex is not particularly limited as long as the specificity can be secured. That is, the magnetic particle can be specifically bound to the primary substance-secondary substance complex by binding two substances that specifically bind to each other to the magnetic particle and the complex, respectively, followed by mixing. Examples of the two substances that specifically bind to each other include avidin and biotin. That is, the magnetic particle can be specifically bound to the complex by, for example, binding avidin to the magnetic particle and binding biotin to the complex. Biotin bound to the complex is preferably bound to, for example, the primary substance or the secondary substance. For example, when biotin is bound to the primary substance, a labeling substance for detection is preferably bound to the secondary substance. On the contrary, a labeling substance may be bound to the primary substance, and biotin may be bound to the secondary substance.

For the two substances that specifically bind to each other, an antigen and an antibody, a lectin and a sugar chain, or a receptor and a ligand can be used instead of avidin and biotin.

### (3) Primary Substance-Secondary Substance Complex Detection Step

In the primary substance-secondary substance complex detection step of the competitive method (D), the primary substance-secondary substance complex on the magnetic particle is detected with a laser. The primary substance-secondary substance complex detection step of the competitive method (D) can be carried out in the same manner as the primary substance-secondary substance complex detection step described in the competitive method (C).

Also in the competitive method (D), examples of the test substance include, but are not limited to, an antigen or an antibody as in the competitive method (C). The antibody immobilization/antigen labeling method and the antigen immobilization/antibody labeling method of the competitive method (C) can be carried out in the same manner except that the order of binding between the magnetic particle and the primary substance (antibody or antigen) is different.

In the competitive method (C), the measurement can be carried out without the washing operation. Meanwhile, in the competitive method (D), after formation of the primary substance-secondary substance complex, the magnetic particle is bound to the complex. Thus, the measurement can be carried out without the washing operation more efficiently than in the case of the competitive method (C).

The types of capture antibody, detection antibody, and the like used in the sandwich method and the competitive method of the present invention are not particularly limited, and examples thereof include a polyclonal antibody, a monoclonal antibody, a recombinant antibody, or an antibody fragment of such an antibody. Examples of the antibody fragment include F(ab′)₂, Fab′, Fab, or Fv. Such an antibody fragment can be obtained by, for example, digesting an antibody with a proteolytic enzyme (e.g., pepsin or papain) by an ordinary method, and then purifying the resulting product by an ordinary protein separation and purification method.

Examples of the test substance in the present invention include, but are not particularly limited to, an antigen or an antibody.

Examples of the antigen include a viral protein, a bacterial protein, or a biomarker (e.g., phosphorylated tau).

Examples of the antibody include an antibody against a virus, an antibody against a bacterium, an autoantibody, or an IgE antibody against a pollinosis antigen or the like.

### Action

In the present invention, the mechanism by which single molecular counting (SMC) measurement can be performed not in a liquid phase but in a solid phase has not been analyzed in detail. However, the mechanism can be presumed as follows.

Usually, in the SMC method, the fluorescent substance is dissociated from the magnetic particle and measured in a liquid phase system so that the magnetic particle does not interfere with the molecule to be measured. The reason for this is probably because the magnetic particle causes interference with a wavelength for measurement with a laser. In the present invention, it has been found that the use of a magnetic particle having a small particle diameter does not cause interference with a wavelength for measurement with a laser. That is, it is presumed that single molecular counting (SMC) measurement can be performed in a solid phase by using a magnetic particle having a particle diameter that does not cause interference with a wavelength for measurement with a laser. When the measurement wavelength is large, the magnetic particle does not cause interference with the measurement wavelength, even if the particle has a relatively large particle diameter. Meanwhile, when the measurement wavelength is small, the magnetic particle having a smaller particle diameter does not cause interference with the measurement wavelength.

### EXAMPLES

Hereinafter, the present invention will be specifically described by way of Examples, but these Examples do not limit the scope of the present invention.

### Example 1

In this Example, tau protein was measured by SMC using magnetic particles having a particle diameter of 100 nm (particle radius: 50 nm).

1 ng of an anti-tau antibody (an antibody obtained by biotinylation of an anti-tau antibody No. 5 available from Kishida Chemical Co., Ltd. with maleimide-PEG11-biotin available from EZ-Link) was immobilized on 40 μL of Therma-Max (TM; JNC; Therma-Max (registered trademark) LA Avidin) at room temperature for 1 hour. Tau protein (recombinant tau-441 available from Sigma-Aldrich) was diluted to 1 pg/mL, 10 pg/mL, 100 pg/mL, 1 ng/mL, or 10 ng/mL, 50 μL of the diluted protein was added, and reaction was allowed to proceed at room temperature for 1 hour. The magnetic particles were washed three times. 20 ng of a labeled antibody (half antibody obtained by protein G purification of a tau antibody A0024 available from DACO, then cleavage of the S—S bond in the hinge region with 1 mM TCEP, and fluorescent labeling of the cleaved product with Alexa Fluor 647 C2 Maleimide) was added, and reaction was allowed to proceed at room temperature for 1 hour. The magnetic particles were washed three times and collected with a magnet. 150 mM NaCl, 50 mM HEPES (pH 7.4) 0.01% SF08 (available from NOF Corporation) was used as a washing buffer or the like.

The collected pellets were measured by SMC with SMCxPRO (Merk).

### Comparative Example 1

In this Comparative Example, tau protein was measured by SMC using magnetic particles having a particle diameter of 2.3 μm.

1 ng of an anti-tau antibody (an antibody obtained by biotinylation of an anti-tau antibody No. 5 available from Kishida Chemical Co., Ltd. with maleimide-PEG11-biotin available from EZ-Link) was immobilized on 5 μL of M270 (Thermo; Dynabeads M270 streptavidin) at room temperature for 1 hour. Tau protein (recombinant tau-441 available from Sigma-Aldrich) was diluted to 1 pg/mL, 10 pg/mL, 100 pg/mL, 1 ng/mL, or 10 ng/mL, 50 μL of the diluted protein was added, and reaction was allowed to proceed at room temperature for 1 hour. The magnetic particles were washed three times. 50 ng of a labeled antibody (half antibody obtained by protein G purification of a tau antibody A0024 available from DACO, then cleavage of the S—S bond in the hinge region with 1 mM TCEP, and fluorescent labeling of the cleaved product with Alexa Fluor 647 C2 Maleimide) was added, and reaction was allowed to proceed at room temperature for 1 hour. The magnetic particles were washed five times. The fluorescent substance was eluted into the liquid phase using 50 μL of 0.1% SDS (pH 3.7). The liquid phase was measured by SMC with SMCxPRO (Merk).

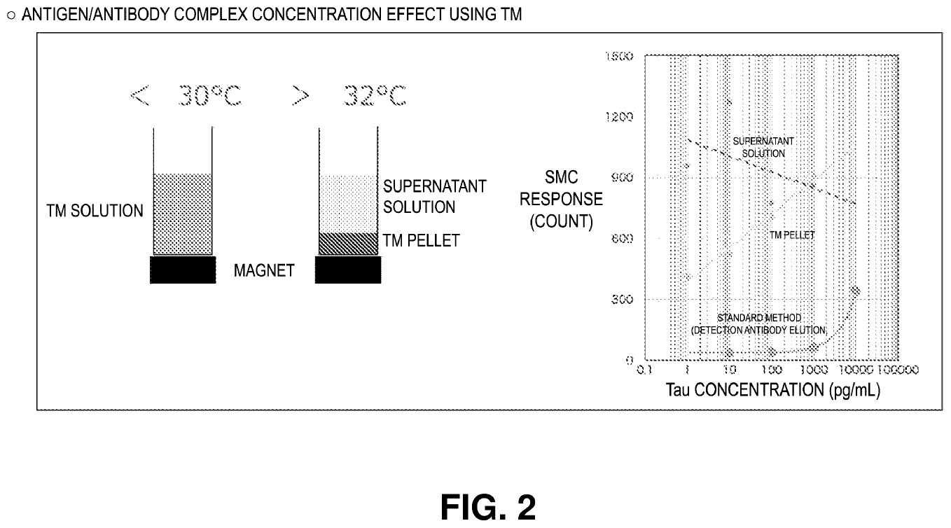

As illustrated in FIG. 2, in Example 1, tau protein was detected down to 1 pg/mL, exhibiting good linearity. In contrast, in the method known in the art of Comparative Example 1, the detection limit of tau protein was 10 ng/ml.

### Example 2

In this Example, the measurement results of optimized tau protein are shown. Specifically, tau protein was diluted to a very low concentration and measured by SMC using magnetic particles having a particle diameter of 100 nm (particle radius: 50 nm).

50 ng of an anti-tau antibody (Fab′ antibody obtained by F(ab′)₂ fragmentation of an anti-pT217 phosphorylated tau antibody p7201 available from Kishida Chemical Co., Ltd. with FabRICATOR (available from Genovis), cleavage of the S—S bond in the hinge region with 1 mM TCEP, and biotinylation with maleimide-PEG11-biotin available from EZ-Link) was immobilized on 70 μL of Therma-Max (TM; JNC; Therma-Max (registered trademark) LA Avidin) at room temperature for 1 hour. Next, 100 μL of 0.0001 fM, 0.001 fM, 0.01 fM, or 0.1 fM solution of a phosphorylated tau protein (tau obtained by phosphorylation of recombinant tau-441 available from Sigma-Aldrich or recombinant tau-441 prepared in-house with active GSK-3bata available from Abcam, active CDK-5 available from Abcam, and ATP available from Thermo) was added, and reaction was allowed to proceed at 4° C. for 1 hour. The magnetic particles were washed three times. 50 ng of a labeled antibody (Fab′ antibody obtained by protein A purification of a tau antibody A0024 available from DACO, F(ab′)₂ fragmentation with Pierce™ Fab Micro Preparation Kit available from Thermo-Fisher, cleavage of the S—S bond in the hinge region with 1 mM TCEP, and fluorescent labeling with Alexa Fluor 647 C2 Maleimide) was added, and reaction was allowed to proceed at 4° C. for 1 hour. The magnetic particles were washed three times and collected with a magnet.

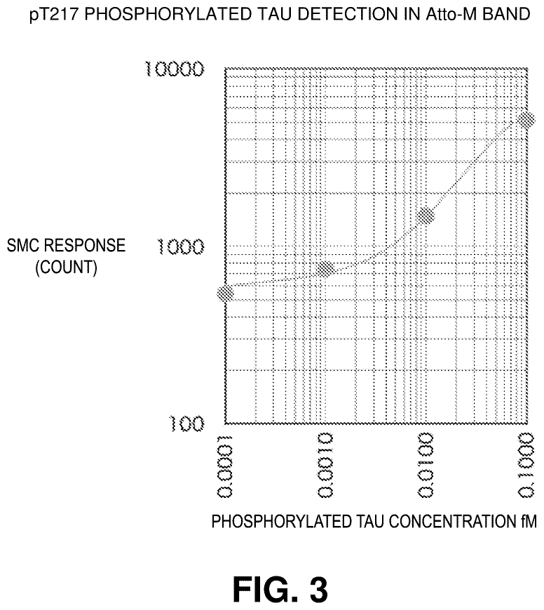

The collected pellets were measured by SMC with SMCxPRO (Merk). As illustrated in FIG. 3, good dilution linearity was exhibited, and it was confirmed that phosphorylated tau at a concentration in the atto-M region can be measured by this technique.

For coating of the capture antibody, 150 mM NaCl, 50 mM HEPES (pH 7.4) 0.1% BSA was used, and 150 mM NaCl, 50 mM HEPES (pH 7.4) 10% TRU blocker (A66800H available from Meridian Biosience) 0.01% Brij 35 (Sigma) was used as a blocking buffer. 350 mM NaCl, 50 mM HEPES (pH 7.4) was used as a washing buffer. 350 mM NaCl, 50 mM HEPES (pH 7.4) 10% TRU blocker (A66800H available from Meridian Biosience) 0.01% Brij 35 (Sigma), 0.1% BSA (Sigma) was used as a solution during action of tau and the detection antibody.

### Example 3

In this Example, the wavelength dependence of absorbance was examined using magnetic particles having a particle diameter of 100 nm (particle radius 50 nm, Therma-Max) and magnetic particles having a particle diameter of 1 μm (particle radius 500 nm, MyOne, Dynabeads). Each of the magnetic substances was suspended in distilled water to such an extent that spontaneous precipitation did not occur. The entire volume was loaded into one well of a 364-well imaging plate (ABB2-00160A available from Aurora Miroplate) and measured with a light absorption spectrum measuring instrument (Nivo 5S available from PerkinElmer).

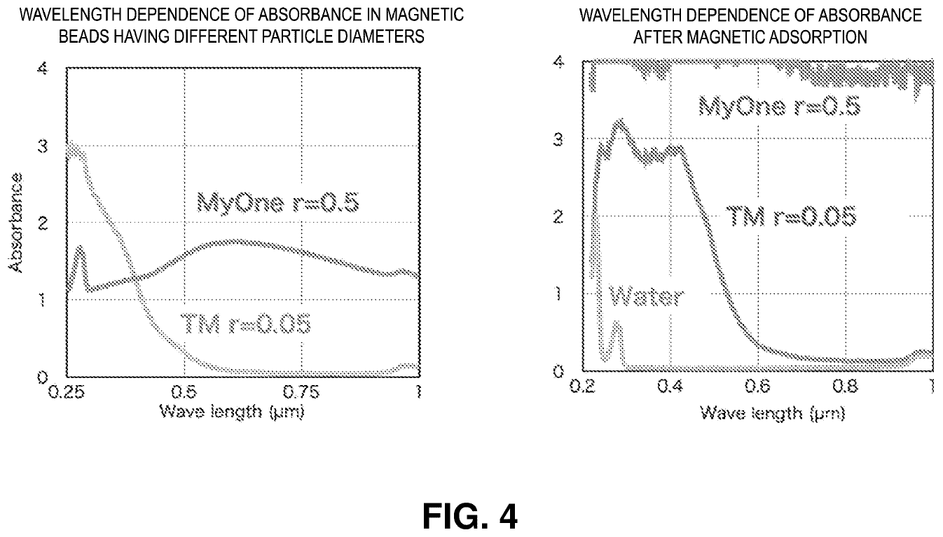

As illustrated on the left side of FIG. 4, in a solution containing the magnetic particles having a particle diameter of 100 nm (total volume 100 μL, Therma-Max:water=1:7 volume ratio) in a suspension state before exposure to magnetism, a sharp decrease in light absorption was observed with an increase in wavelength, and there was almost no absorption of incident light having a wavelength of 600 nm (0.6 μm) or more. The results indicated that light observation can be performed in this range. In contrast, in a solution containing the magnetic particles having a particle diameter of 1000 nm (1 μm) (total volume 100 μL, MyOne:water=1:49 volume ratio), such a sharp attenuation of light absorption was not observed, and strong light absorption was measured over the entire wavelength range of 0.25 to 1000 nm for measurement, indicating that optical measurement with light of all wavelengths is difficult by using this type of beads.

The right side of FIG. 4 illustrates the light absorption spectra of the pellets prepared by magnetically capturing each of the magnetic substances. A solution containing the magnetic particles having a particle diameter of 100 nm (total volume 100 μL, Therma-Max:water=1:2 volume ratio), a solution containing the magnetic particles having a particle diameter of 1000 nm (1 μm) (total volume 100 μL, MyOne:water=1:7 volume ratio), and a solvent (distilled water, total volume 100 μL) were prepared, and each of these was loaded into a 362-well imaging plate. Each of the magnetic substances was deposited on the bottom of the plate to prepare pellets, and the absorbance spectrum was measured. The results indicated that the features observed in the suspension are substantially maintained in the pellets as well; specifically, in the case of the pellets containing the magnetic particles having a particle diameter of 100 nm, there is almost no absorption of incident light having a wavelength of 600 nm (0.6 μm) or more, and light observation can be performed in this range; and in the case of the pellets containing the magnetic particles having a particle diameter of 1000 nm (1 μm), strong light absorption is measured over the entire wavelength range of 0.25 to 1000 nm for measurement, and optical measurement with light of all wavelengths is difficult by using this type of beads.

### Example 4

In this Example, tau protein was measured by SMC without washing by the sandwich method (B) using magnetic particles having a particle diameter of 100 nm (particle radius: 50 nm).

The phosphorylated tau protein (prepared in Example 2) was diluted to 10 ag/mL, 100 ag/mL, 100 ag/mL, 1 fg/mL, or 10 fg/mL, and 10 μL of the diluted protein was added to a mixed liquid (100 μL) containing an anti-tau antibody (Kishida phosphorylated tau antibody p7204 (Fab, biotinylated) (10 μg/100 μL buffer) as a capture antibody and an anti-tau antibody (Kishida tau antibody Tau1-20) (Fab, Alexa 647) (20 μg/100 μL buffer) as a detection antibody, and reaction was allowed to proceed at room temperature for 2 hours.

Therma Max (TM; JNC; avidinated magnetic particles) exposed to a blocking buffer (300 mM NaCl, 50 mM Hepes, 0.5% BSA, 0.01% Brij 35, 1% TRU Ultra) (room temperature for 1 hour or more) was used as a stock liquid (5 mg/mL). In this Example, immediately before use, the TM stock liquid was dispensed into a 96-well plate (Watson, 537-96-TP) (50 μL for each well), the beads were magnetically concentrated at 37° C. to remove the buffer, then 100 μL of the mixed liquid was added, and reaction was allowed to proceed for 1 hour. Thereafter, the reaction liquid containing TM was heated to 37° C. (for 3 minutes) and transferred to a 394-well imaging plate (Aurora Microplates ULB SQ/EB) at a high-temperature state. Pellets were formed on the bottom of the plate with a magnet (Thistile Scientific, VP 771G-4AAZM-1), and SMC was performed with SMCxPRO (Merk). In addition, the tau protein was decreased to ¼, and the same operation was performed.

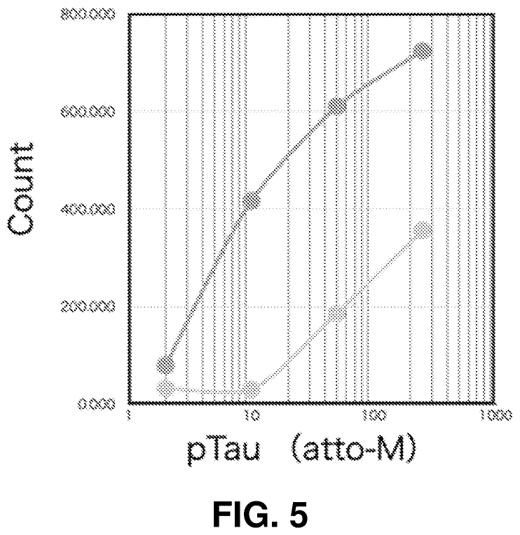

As illustrated in FIG. 5, high measurement sensitivity was exhibited even without washing.

### INDUSTRIAL APPLICABILITY

The method for detecting a test substance in a specimen and the magnetic particle of the present invention can be used for highly sensitive single molecular counting (SMC) measurement.

1. A method for detecting a test substance in a specimen, the method being
(A) a sandwich method comprising:
(1) bringing a specimen into contact with a magnetic particle on which a primary substance is immobilized and which has a particle diameter that does not cause interference with a wavelength for measurement with a laser, to form a primary substance-test substance complex of the primary substance and a test substance in the specimen;
(2) bringing the primary substance-test substance complex into contact with a secondary substance, to form a primary substance-test substance-secondary substance complex; and
(3) detecting the primary substance-test substance-secondary substance complex on the magnetic particle with a laser;
(B) a sandwich method comprising:
(1) bringing a primary substance into contact with a test substance in a specimen and a secondary substance, to form a primary substance-test substance-secondary substance complex;
(2) binding the primary substance-test substance-secondary substance complex to a magnetic particle having a particle diameter that does not cause interference with a wavelength for measurement with a laser; and
(3) detecting the primary substance-test substance-secondary substance complex on the magnetic particle with a laser;
(C) a competitive method comprising:
(1) bringing a specimen and a secondary substance into contact with a magnetic particle on which a primary substance is immobilized and which has a particle diameter that does not cause interference with a wavelength for measurement with a laser, to form a primary substance-secondary substance complex of the primary substance and the secondary substance; and
(2) detecting the primary substance-secondary substance complex on the magnetic particle with a laser; or
(D) a competitive method comprising:
(1) bringing a primary substance into contact with a specimen and a secondary substance, to form a primary substance-secondary substance complex of the primary substance and the secondary substance;
(2) binding the primary substance-secondary substance complex to a magnetic particle having a particle diameter that does not cause interference with a wavelength for measurement with a laser; and
(3) detecting the primary substance-secondary substance complex on the magnetic particle with a laser.

2. The method for detecting a test substance according to claim 1, wherein, in the sandwich method (A) or (B), the primary substance is a capture antibody, the test substance is an antigen in the specimen, and the secondary substance is a detection antibody conjugated with a label that is detectable by the laser.

3. The method for detecting a test substance according to claim 1, wherein, in the sandwich method (A) or (B), the primary substance is an antigen, the test substance is an antibody in the specimen, and the secondary substance is a detection antibody conjugated with a label that is detectable by the laser.

4. The method for detecting a test substance according to claim 1, wherein, in the competitive method (C) or (D), the primary substance is a capture antibody, the test substance is an antigen or antibody in the specimen, and the secondary substance is a standard antigen conjugated with a label that is detectable by the laser.

5. The method for detecting a test substance according to claim 1, wherein, in the competitive method (C) or (D), the primary substance is an antigen, the test substance is an antigen or antibody in the specimen, and the secondary substance is a detection antibody conjugated with a label that is detectable by the laser.

6. The method for detecting a test substance according to claim 1, wherein the detection with a laser uses a laser confocal microscope.

7. The method for detecting a test substance according to claim 1, wherein the particle diameter is from 20 to 500 nm.

8. A magnetic particle having a particle diameter that does not cause interference with a wavelength for measurement with a laser, the magnetic particle being for use in a method for detecting a test substance in a specimen, the method being a sandwich method (A) or (B), or a competitive method (C) or (D), the method comprising steps described below:
(A) a sandwich method comprising:
(1) bringing a specimen into contact with the magnetic particle on which a primary substance is immobilized, to form a primary substance-test substance complex of the primary substance and a test substance in the specimen;
(2) bringing the primary substance-test substance complex into contact with a secondary substance, to form a primary substance-test substance-secondary substance complex; and
(3) detecting the primary substance-test substance-secondary substance complex on the magnetic particle with a laser;
(B) a sandwich method comprising:
(1) bringing a primary substance into contact with a test substance in a specimen and a secondary substance, to form a primary substance-test substance-secondary substance complex;
(2) binding the primary substance-test substance-secondary substance complex to the magnetic particle; and
(3) detecting the primary substance-test substance-secondary substance complex on the magnetic particle with a laser;
(C) a competitive method comprising:
(1) bringing a specimen and a secondary substance into contact with the magnetic particle on which a primary substance is immobilized, to form a primary substance-secondary substance complex of the primary substance and the secondary substance; and
(2) detecting the primary substance-secondary substance complex on the magnetic particle with a laser; or
(D) a competitive method comprising:
(1) bringing a primary substance into contact with a specimen and a secondary substance, to form a primary substance-secondary substance complex of the primary substance and the secondary substance;
(2) binding the primary substance-secondary substance complex to the magnetic particle; and
(3) detecting the primary substance-secondary substance complex on the magnetic particle with a laser.

9. The magnetic particle according to claim 8, wherein, in the sandwich method (A) or (B), the primary substance is a capture antibody, the test substance is an antigen in the specimen, and the secondary substance is a detection antibody conjugated with a label that is detectable by the laser.

10. The magnetic particle according to claim 8, wherein, in the sandwich method (A) or (B), the primary substance is an antigen, the test substance is an antibody in the specimen, and the secondary substance is a detection antibody conjugated with a label that is detectable by the laser.

11. The magnetic particle according to claim 8, wherein, in the competitive method (C) or (D), the primary substance is a capture antibody, the test substance is an antigen or antibody in the specimen, and the secondary substance is a standard antigen conjugated with a label that is detectable by the laser.

12. The magnetic particle according to claim 8, wherein, in the competitive method (C) or (D), the primary substance is an antigen, the test substance is an antigen or antibody in the specimen, and the secondary substance is a detection antibody conjugated with a label that is detectable by the laser.

13. The magnetic particle according to claim 8, wherein the detection with a laser uses a laser confocal microscope.

14. The magnetic particle according to claim 8, wherein the particle diameter is from 20 to 500 nm.

15. The method for detecting a test substance according to claim 1, further comprising collecting the magnetic particle with a magnet before the detecting step.
